(19) United States
(12) Patent Application Publication
(10) Pub. No.: US 2015/0306315 A1
(43) Pub. Date: Oct. 29, 2015

(54) SYRINGE PUMP WITH REPLACEABLE SYRINGE CARTRIDGES

(71) Applicant: NORGREN KLOEHN, INC., Las Cegas, NV (US)

(72) Inventors: Sean Robert Birnbaum, Anchorage, AK (US); Brian Sweeney, Las Vegas, NV (US); Timmy HO, Las Vegas, NV (US); Walter Kish, Las Vegas, NV (US); John Fulton, Plymouth, MA (US)

(21) Appl. No.: 14/386,258

(22) PCT Filed: Mar. 14, 2013
(86) PCT No.: PCT/US13/31488

§ 371 Date: Sep. 18, 2014

Related U.S. Application Data
(60) Provisional application No. 61/613,031, filed on Mar. 20, 2012.

(51) Int. Cl. A61M 5/20

(52) U.S. Cl. CPC A61M 5/20; A61M 2205/12; A61M 2207/00; A61M 2205/3327

(57) ABSTRACT

A syringe system (100) is provided. The syringe system comprises a pump assembly (101) including a movable actuation rod (440) and a plunger contact (445) coupled to an end of the movable actuation rod (440). The syringe system also comprises a cartridge assembly (102). The cartridge assembly (102) includes a manifold (105) removably coupled to the pump assembly (101) and a syringe cartridge (107) positioned within the manifold (105) and including a plunger assembly (330) movable with respect to the manifold (105) upon actuation of the actuation rod (440).

### TECHNICAL FIELD

The embodiments described below relate to, syringe pumps, and more particularly, to syringe pumps with replaceable syringe cartridges.

### BACKGROUND OF THE INVENTION

Syringes generally include a plunger assembly that moves within a fluid barrel. The plunger can draw in a fluid when the plunger is retracted and can dispense the fluid when the plunger is pushed into the bore. Syringes are known for their precise fluid control, especially at low fluid rates/volumes and thus, have received great success in the medical and laboratory fields. Typically, the syringe includes a single fluid port, wherein the fluid is drawn into and expelled from the fluid barrel through the single port.

Although the syringes can be controlled manually, they are often utilized with electronic syringe pumps to provide an automated system. For example, in the medical field, the syringe pumps can provide automated dosing to a patient. In some situations, the syringe pump can form part of a larger syringe pump manifold system wherein multiple syringes are coupled to the manifold. Each syringe may include its own pump or a syringe pump may control multiple syringes. The syringe pump is usually a motorized device which holds the syringe and moves the syringe plunger at a specified rate, which may be programmed by the user, external controller, processing system, etc.

In many situations, syringes are reusable and may be filled and subsequently emptied multiple times. Due in part to the continuous sliding between the plunger and the fluid barrel, the syringe often fails before the pump fails. However, while the syringe pump may still be functioning satisfactorily, the prior art syringe systems replace the entire unit due to the faulty syringe. Further, there are many situations where the syringe's failure causes fluid leakage that destroys the pump's electronics thereby causing failure of the pump as well. This often occurs when the syringe's plunger seal fails and allows fluid to leak past the plunger towards the pump electronics. Typically, the failed seal is not detected until it is too late.

Although replaceable syringe cartridges are known in the prior art, many of these cartridges do not provide satisfactory solutions. For example, with automated systems, it is often very difficult to maintain the small tolerances from one syringe cartridge to the next in order to preserve accurate fluid control. Further, many of the existing systems on the market do not adequately protect the pump electronics in the event of a syringe failure.

The embodiments described below overcome these and other problems and an advance in the art is achieved. The embodiments described below provide a replaceable syringe pump cartridge that can adequately seal the fluid away from the pump electronics while also maintaining small tolerances from one syringe cartridge to another to ensure accurate and repeatable fluid dispensing.

### SUMMARY OF THE INVENTION

A syringe system is provided according to an embodiment. The syringe system comprises a pump assembly including a movable actuation rod and a plunger contact coupled to an end of the movable actuation rod. According to an embodiment, the syringe system also comprises a cartridge assembly. The cartridge assembly includes a manifold removably coupled to the pump assembly and a syringe cartridge positioned within the manifold. According to an embodiment, the syringe cartridge includes a plunger assembly movable with respect to the manifold upon actuation of the actuation rod.

A cartridge assembly for a syringe system is provided according to an embodiment. The cartridge assembly comprises a manifold including one or more fluid ports and a syringe housing positioned within the manifold. According to an embodiment, the cartridge assembly further comprises a fluid barrel positioned within the syringe housing and in fluid communication with the one or more fluid ports and a plunger assembly movable within the fluid barrel to draw fluid into and expel fluid out of the fluid barrel.

A method for forming a syringe system is provided according to an embodiment. According to an embodiment, the method comprises positioning a movable actuation rod and a plunger contact within a pump assembly and positioning a syringe cartridge within a manifold, the syringe cartridge comprising a plunger assembly movable with respect to the manifold. According to an embodiment, the method further comprises removably coupling the manifold to the pump assembly such that actuation of the actuation rod moves the plunger assembly.

### ASPECTS

According to an aspect, a syringe system comprises:
a pump assembly including a movable actuation rod and a plunger contact coupled to an end of the movable actuation rod;
a cartridge assembly including:
   a manifold removably coupled to the pump assembly; and
   a syringe cartridge positioned within the manifold and including a plunger assembly movable with respect to the manifold upon actuation of the actuation rod.

Preferably, the syringe cartridge further comprises a syringe housing and a fluid barrel positioned at least partially within the syringe housing.

Preferably, the plunger assembly comprises a plunger button contacting the plunger contact, a plunger rod coupled to the plunger button, and a plunger tip coupled to the plunger rod, with the plunger tip forming a substantially fluid-tight seal with the fluid barrel.

Preferably, the syringe system further comprises a manifold sealing member coupled to the fluid barrel and forming a substantially fluid-tight seal with the manifold and the syringe housing.

Preferably, the syringe system further comprises a fluid channel formed in the manifold sealing member and in fluid communication with one or more fluid ports formed in the manifold.

Preferably, the syringe system further comprises a plunger guide received by the syringe housing and receives the plunger rod by a sliding radial contact to maintain an axial alignment of the plunger assembly.

Preferably, the syringe system further comprises an auxiliary flow path defined by an auxiliary flow channel between the plunger rod and the fluid barrel, a fluid port formed in the plunger guide, and a fluid port formed in the manifold.

Preferably, the syringe system further comprises one or more coupling tabs formed on the pump assembly and one or more corresponding grooves formed in the manifold.

Preferably, the syringe system further comprises a ramped surface coupled to the actuation rod positioned to contact a pump housing at an extended stroke position.

Preferably, the plunger contact is positioned such that the plunger contact is substantially flush with the pump housing when the actuation rod is at the extended stroke position.

Preferably, the syringe system further comprises a position sensor configured to determine at least an extended position of the actuation rod.

Preferably, the position sensor comprises an optical position sensor and includes a sensor flag coupled to the actuation rod.

Preferably, the plunger contact comprises a magnet.

According to another aspect, a cartridge assembly for a syringe system comprises:
a manifold including one or more fluid ports;
a syringe housing positioned within the manifold;
a fluid barrel positioned within the syringe housing and in fluid communication with the one or more fluid ports; and
a plunger assembly movable within the fluid barrel to draw fluid into and expel fluid out of the fluid barrel.

Preferably, the plunger assembly comprises a plunger tip, a plunger rod, and a plunger button, wherein the plunger button is substantially flush with the manifold when the plunger assembly is in a fully extended position.

Preferably, the cartridge assembly further comprises a plunger guide received by the syringe housing and receives the plunger rod by a sliding radial contact to maintain an axial alignment of the plunger assembly.

Preferably, the cartridge assembly further comprises an auxiliary fluid path defined by an auxiliary flow channel between the plunger assembly and the fluid barrel, a fluid port formed in the plunger guide, and a fluid port formed in the manifold.

Preferably, the cartridge assembly further comprises a manifold sealing member coupled to the fluid barrel and forming a substantially fluid-tight seal with the manifold and the syringe housing.

Preferably, the cartridge assembly further comprises a fluid channel formed in the manifold sealing member and in fluid communication with one or more fluid ports formed in the manifold.

According to another aspect, a method for forming a syringe system comprises steps of:
positioning a movable actuation rod and a plunger contact within a pump assembly;
positioning a syringe cartridge within a manifold, the syringe cartridge comprising a plunger assembly movable with respect to the manifold; and
removably coupling the manifold to the pump assembly such that actuation of the actuation rod moves the plunger assembly.

Preferably, the step of positioning the syringe cartridge comprises inserting a fluid barrel into a syringe housing and coupling the fluid barrel to the manifold to create a seal.

Preferably, the method further comprises a step of aligning a fluid channel formed in the fluid barrel with one or more fluid ports formed in the manifold.

Preferably, the plunger assembly comprises a plunger tip forming a substantially fluid-tight seal with the fluid barrel, a plunger rod coupled to the plunger tip, and a plunger button coupled to the plunger rod, wherein the step of removably coupling the manifold positions the plunger button in contact with the plunger contact.

Preferably, the method further comprises a step of positioning a plunger guide within the syringe housing and by a sliding radial contact, positions the plunger rod within the plunger guide to maintain axial alignment of the plunger assembly.

Preferably, the method further comprises a step of defining an auxiliary flow channel between the plunger rod and the fluid barrel, which is in fluid communication with a fluid port formed in the plunger guide and a fluid port formed in the manifold.

Preferably, the step of removably coupling the manifold comprises engaging one or more coupling tabs extending from the pump assembly with one or more corresponding grooves formed in the manifold.

Preferably, the step of positioning the movable actuation rod comprises actuating the actuation rod to an extended position and coupling the plunger contact to the actuation rod such that the plunger contact is substantially flush with a housing of the pump assembly.

Preferably, the method further comprises a step of coupling a portion of a position sensor to the actuation rod to detect at least an extended position of the actuation rod.

### BRIEF DESCRIPTION OF THE DRAWINGS

FIG. 1 shows a syringe system according to an embodiment.

FIG. 2 shows a partially exploded view of the syringe system according to an embodiment.

FIG. 3 shows an exploded view of a syringe cartridge according to an embodiment.

FIG. 4 shows a cross-sectional view of a portion of a pump assembly of the syringe system according to an embodiment.

FIG. 5 shows a cross-sectional view of a portion of the syringe system according to an embodiment.

### DETAILED DESCRIPTION OF THE INVENTION

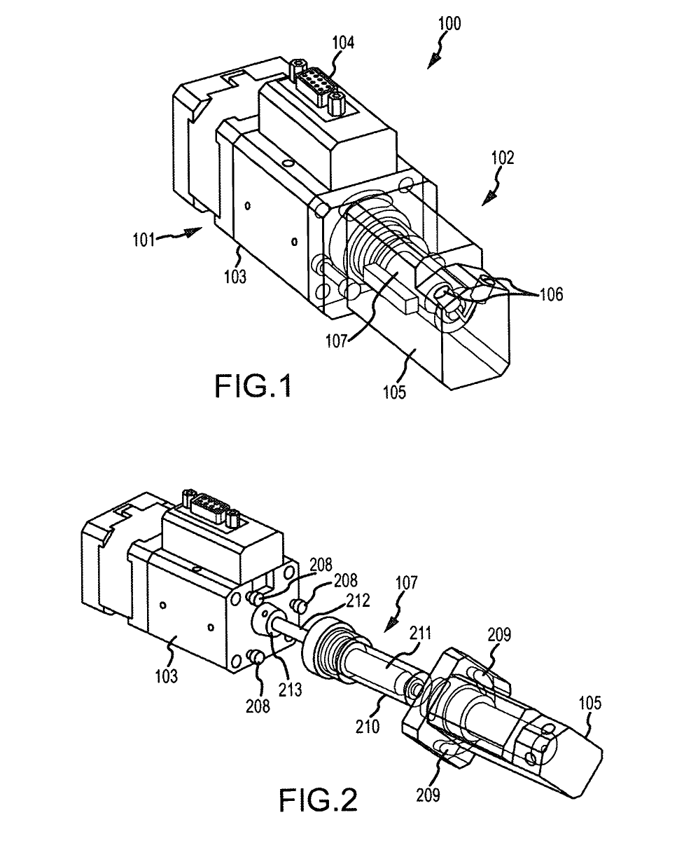

FIGS. 1-5 and the following description depict specific examples to teach those skilled in the art how to make and use the best mode of embodiments of a syringe system. For the purpose of teaching inventive principles, some conventional aspects have been simplified or omitted. Those skilled in the art will appreciate variations from these examples that fall within the scope of the present description. Those skilled in the art will appreciate that the features described below can be combined in various ways to form multiple variations of the syringe system. As a result, the embodiments described below are not limited to the specific examples described below, but only by the claims and their equivalents.

FIG. 1 shows a syringe system 100 according to an embodiment. The syringe system 100 comprises a pump assembly 101 and a cartridge assembly 102. The pump assembly 101 comprises a housing 103 and an electrical module 104. The electrical module 104 can provide electrical communication with an external computer, processing system, controller, etc. The electrical module 104 can also provide power to the pump assembly 101. The pump assembly 101 can actuate and control fluid dispensing of the syringe system 100.

According to an embodiment, the cartridge assembly 102 comprises a manifold 105. The manifold 105 comprises one or more fluid ports 106. The fluid ports 106 can provide fluid communication between a syringe cartridge 107 and a desired device (not shown) fluidly coupled to the fluid ports 106, such as a manifold assembly. The device may comprise a wide variety of devices, and the particular device the syringe system 100 provides fluid to should in no way limit the scope of the present embodiment. Although the syringe cartridge 107 is visible through the partially transparent manifold 105, in other embodiments, the manifold 105 may be opaque and the syringe cartridge 107 may not be visible. However, providing at least a partially transparent manifold 105 may aid a user in detecting a syringe failure. Additionally, the partially transparent manifold 105 may allow a user to visually identify an approximate amount of fluid remaining in the syringe cartridge 107. In addition to or as an alternative to providing the at least partially transparent manifold 105, in some embodiments, a separate viewing window may be cut into the manifold 105 to allow direct viewing access of the syringe cartridge 107.

FIG. 2 shows a partially exploded view of the syringe system 100 according to an embodiment. In FIG. 2 the manifold 105 has been decoupled from the pump assembly housing 103. As can be seen in FIG. 2, in some embodiments, the pump assembly housing 103 may include one or more coupling tabs 208, which can engage corresponding grooves 209 formed in the manifold 105 to removably couple the components to one another. Two of the grooves 209 are visible in FIG. 2. In some embodiments, the coupling tabs 208 and the grooves 209 may form a snap-fit arrangement to positively couple the components. According to an embodiment, the grooves 209 may include inclined ramps which can pull the components together as the coupling tabs 208 are received by the grooves 209 and slide up the ramps. For example, the coupling tabs 208 can initially be received by the grooves 209 and then when the manifold 105 is rotated with respect to the pump housing 103 to move the coupling tabs 208 to a locked position, the two components can be pulled together.

It should be appreciated that while the coupling tabs 208 and grooves 209 are shown in the embodiments depicted, other components may be utilized to couple the manifold 105 to the pump assembly housing 103. For example, in other embodiments, the syringe system 100 may utilize bolts, adhesives, magnets, etc. Consequently, the claims that follow should not be limited to requiring the coupling tabs 208 and the grooves 209. However, the use of the coupling tabs 208 and the grooves 209 allows for a tool-less coupling and de-coupling of the manifold 105 and the pump assembly housing 103.

Also more visible in FIG. 2 is the syringe cartridge 107. According to an embodiment, the syringe cartridge 107 comprises an outer syringe housing 210 and an inner fluid barrel 211. Although the syringe cartridge 107 is shown as substantially cylindrical, other shapes may be used. According to an embodiment, there may be a space between the radially outer surface of the fluid barrel 211 and the radially inner surface of the syringe housing 210. The space can be provided such that the syringe housing 210 can receive more than one sized fluid barrel 211. Therefore, the syringe cartridge 107 can accommodate fluid barrels of different volumes while maintaining a consistent outer profile due to the syringe housing 210.

According to the embodiment shown, the syringe cartridge 107 further comprises a plunger rod 212 and a plunger button 213, which are shown extending from the syringe housing 210. As can be appreciated, in response to a signal from a controller, the pump assembly can act on the plunger button 213 to actuate the plunger rod 212 in a first or a second direction to extend or withdraw the plunger rod 212. As those skilled in the art will recognize, actuation of the plunger rod 212 can dispense fluid from or draw fluid into the fluid barrel 211.

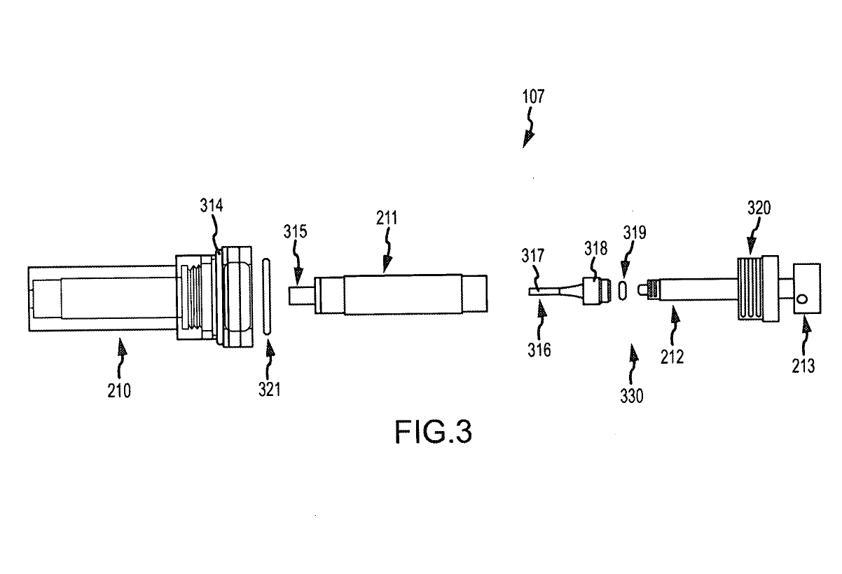

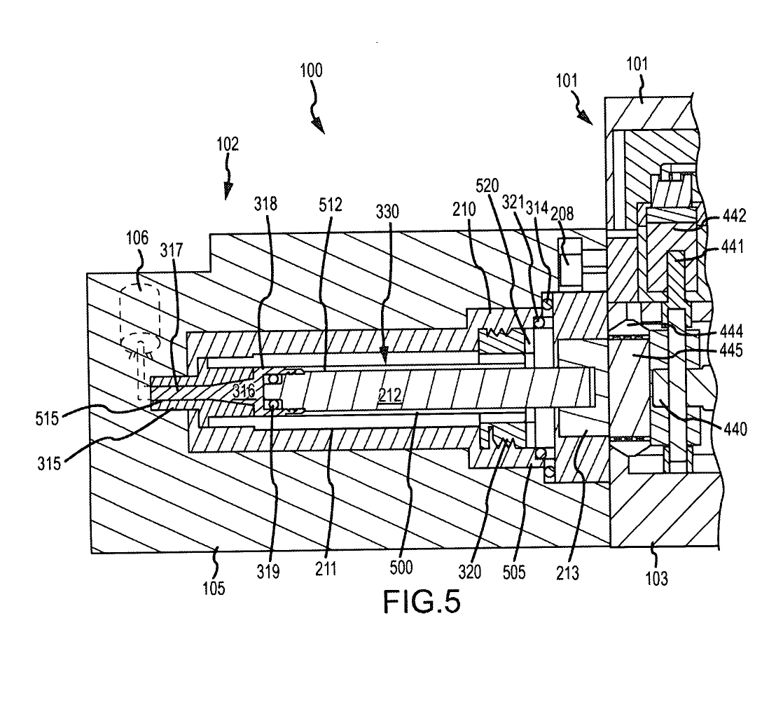

FIG. 3 shows an exploded view of the syringe cartridge 107 according to an embodiment. According to the embodiment shown, the syringe housing 210 can receive at least a portion of the fluid barrel 211. In some embodiments, the syringe housing 210 can be formed from a plastic or acrylic material while the fluid barrel 211 can be formed from glass, for example. However, other materials may be used and the particular material used should in no way limit the scope of the present embodiment. According to the embodiment shown, the syringe housing 210 can receive an outer sealing member 314. The outer sealing member 314 can be provided to form a substantially fluid-tight seal between the manifold 105 and the syringe housing 210 (See FIG. 5). In some embodiments, the sealing member 314 can be utilized to prevent fluid from reaching the pump assembly 101 in the event of a syringe failure. The outer sealing member 314 is discussed in more detail below.

According to an embodiment, the fluid barrel 211 can include a manifold sealing member 315. As can be seen in more detail in FIG. 5, a portion of the manifold sealing member 315 can extend from the syringe housing 210. The manifold sealing member 315 can comprise a compressible material that can form a substantially fluid-tight seal with the manifold 105 and the syringe housing 210 when the syringe system 100 is assembled. In other embodiments, the manifold sealing member 315 can comprise a separate removable sealing member. However, in the embodiment shown, fewer components are required to form the fluid-tight seal as the manifold sealing member 315 can provide the fluid-tight seal while also defining a portion of the fluid flow path (See FIG. 5).

According to the embodiment shown, the syringe cartridge 107 further includes a plunger tip 316. The plunger tip 316 can be coupled to the plunger rod 212. A sealing member 319 can be positioned inside the second end 318 to provide an outward force on the second end 318 to increase the sealing capability between second end 318 and the fluid barrel 211. The plunger tip 316, the plunger rod 212, and the plunger button 213 together form a movable plunger assembly 330, which dispenses the fluid within the fluid barrel 211 when actuated by the pump assembly 101. The plunger tip 316 can include a first end 317 that can be at least partially received by the manifold sealing member 315. The plunger tip 316 can further include a second end 318 that can form a substantially fluid-tight seal with the fluid barrel 211. Therefore, in some embodiments, the second end 318 of the plunger tip 316 can be formed from a partially compressible material, which can partially compress to form the fluid-tight seal with the interior of the fluid barrel 211. Therefore, in some embodiments, the second end 318 of the plunger tip 316 can be slightly larger than the inner circumference of the fluid barrel 211 in the uncompressed state. The reduced size of the fluid barrel 211 can compress the second end 318 of the plunger tip 316 to form the fluid-tight seal. Often, the sliding between the plunger tip 316 and the fluid barrel 211 causes the plunger tip 316 to wear and fail before many of the other components of the syringe system 100. Wear between the two components can cause fluid leakage past the plunger tip 316.

Although the description above uses the second end 318 of the plunger tip 316 to provide the sealing mechanism, in other embodiments, an additional sealing member may be provided to create the fluid-tight seal. In other words, in other embodiments, the second end 318 may be smaller than the fluid barrel 211 and a separate sealing member can provide the needed fluid-tight seal. Such embodiments may extend the life of the plunger tip 316 by allowing replacement of the removable sealing member.

Also shown in FIG. 3 is a plunger guide 320. The plunger guide 320 can receive at least a portion of the plunger rod 212. As shown in FIG. 3, the plunger rod 212 is extending through the plunger guide 320. The plunger rod 212 can slide within the plunger guide 320. Therefore, the plunger guide 320 can help maintain proper axial alignment of the plunger rod 212 during actuation. As can be seen, the plunger button 213 cannot move past the plunger guide 320 because the plunger button 213 is generally larger than the internal opening in the plunger guide 320. According to an embodiment, the plunger guide 320 can also be received by the syringe housing 210. For example, the plunger guide 320 in some embodiments can be coupled via a threaded connection to the syringe housing 210. In some embodiments, a sealing member 321 can provide a substantially fluid-tight seal between the syringe housing 210 and the plunger guide 320. Therefore, the outer surface of the plunger guide 320 along with the sealing member 321 can create a friction fit with the inner surface of the syringe housing 210 and axial alignment of the plunger guide 320 can be maintained. Maintaining axial alignment of the plunger guide 320 reduces the uneven wear of the plunger tip 316 as it slides within the fluid barrel 211 to extend the life of the syringe cartridge 107.

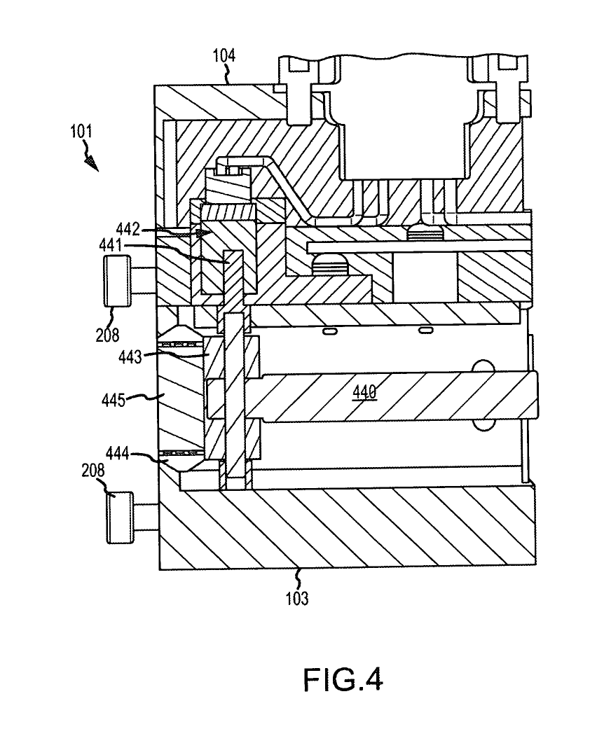

FIG. 4 shows a cross-sectional view of a section the pump assembly 101 according to an embodiment. Although the right side of the pump assembly 101 is not visible, it comprises well-known components that are not important for describing the present embodiment. According to the embodiment shown in FIG. 4, the pump assembly 101 comprises an actuation rod 440, which is coupled at one end to a motor, solenoid, fluid supply, etc., which is not shown, in order to actuate the actuation rod 440 between first and second end positions. In the position shown, the actuation rod 440 is in the fully extended position. As those skilled in the art can readily understand, the retracted position would move the actuation rod 440 to the right according to the orientation shown in FIG. 4. The particular method used to actuate the actuation rod 440 is not important for purposes of the present embodiment and will depend on the particular pump chosen. Therefore, the particular actuation method should in no way limit the scope of the present embodiment.

According to an embodiment, the actuation rod 440 is coupled to a sensor flag 441. The sensor flag 441 can comprise a portion of a position sensor 442. In some embodiments, the position sensor 442 can comprise an optical sensor. However, other types of position sensors may be utilized. According to an embodiment, the sensor flag 441 can interact with an optical source and detector (not shown) to signal at least an end position of the actuation rod 440. For example, upon receiving a signal from the position sensor 442, the actuation force provided to the actuation rod 440 may be removed. Additionally, a user or operator may be alerted that the actuation rod 440 has reached an end position.

In other embodiments, the position sensor 442 may be omitted and the controller can remove the actuation force provided to the actuation rod 440 once a threshold counter force is experienced. For example, in the embodiment shown, the counter force can be provided due to abutment with the housing 103. Therefore, the pump assembly 101 should not be limited to requiring the position sensor 442.

In addition to the sensor flag 441, the actuation rod 440 is further coupled to a hub 443. According to an embodiment, the hub 443 includes a ramped surface 444 that contacts the body 103 of the pump assembly 101 to provide a positive stop for the actuation rod 440. According to an embodiment, the ramped surface 444 may comprise a separate component from the hub 443 or the ramped surface 444 may be integrally formed as part of the hub 443. As can be seen, the actuation rod 440 cannot move further to the left as shown in FIG. 4 due to the abutment of the hub 443 with the body 103.

According to an embodiment, the hub 443 is also provided to receive a plunger contact 445. According to an embodiment, the plunger contact 445 can be held in the hub 443 according to well-known techniques, such as adhesives, brazing, bonding, mechanical fasteners, etc. According to an embodiment, the plunger contact 445 can contact the plunger button 213. In some embodiments, the plunger contact comprises a magnet. However, in other embodiments, the plunger contact 445 may include an adhesive or some other non-rigid contact surface. The plunger contact 445 should be able to apply an actuation pushing force on the plunger button 213 as well as provide an actuation pulling force on the plunger button 213, i.e., actuate the plunger assembly 330 in both directions. Therefore, if the plunger button 213 comprises a magnetic material, the plunger contact 445 can push and pull the plunger button 213 to extend and retract the plunger rod 212. Furthermore, once the actuation rod 440 and thus, the plunger contact 445 is in the position shown in FIG. 4, the syringe cartridge 107 can easily be removed from the syringe system 100 once a threshold force is provided that can overcome the magnetic attraction between the plunger contact 445 and the plunger button 213.

Additionally, in many prior art syringe systems, it was difficult to accurately align the plunger assembly in a repeatable manner with low tolerances to obtain accurate fluid distribution. However, in the present embodiment, the plunger contact 445 can ensure accurate and repeatable positioning of the plunger button 213 and thus, the entire plunger assembly 330. For example, in one embodiment, once the actuation rod 440 is in the fully extended position, the plunger contact 445 can be precisely positioned to ensure the outer face (left side as shown in the figures) of the plunger contact 445 is flush with the pump body 103. Therefore, any tolerance issues of the actuation rod 440 with the body can be corrected by the plunger contact 445. Using this technique, tolerance accumulation (accumulation in errors of the precise positioning of the components) between the various components of the pump assembly 101 can be eliminated or substantially reduced by the positioning of the plunger contact 445.

Furthermore, by utilizing the plunger contact 445 rather than a rigid mechanical coupling between the actuation rod 440 and the plunger button 213, any small misalignments or loss of concentricity between the plunger button 213 and the actuation rod 440 will not create additional stress or uneven wear. Rather, the actuation rod 440 can still apply axial movement to the plunger button 213 without applying a substantial force in a direction perpendicular to the axial direction. For example, in some embodiments, the plunger button 213 may be able to slide against the plunger contact 445 in directions perpendicular to the axial movement with respect to the plunger contact 445 to self-correct misalignments.

FIG. 5 shows a cross-sectional view of a portion of the syringe system 100 according to an embodiment. In the embodiment shown in FIG. 5, the right end of the pump assembly 101 is not visible; however, the portion not shown comprises well-known components and therefore, is omitted for simplicity.

According to the embodiment shown in FIG. 5, the plunger assembly 330 is in the fully extended position. This is due to the actuation of the actuation rod 440. In the fully extended position, the first end 317 of the plunger tip 316 is received by the manifold sealing member 315. More specifically, the plunger tip 316 is received by a fluid channel 515 defined by the manifold sealing member 315. As can be appreciated, the fluid channel 515 forms a portion of the fluid pathway as mentioned above.

According to an embodiment, the fluid channel 515 is in fluid communication with the one or more fluid ports 106. The fluid channel 515 is also in fluid communication with an interior of the fluid barrel 211. Consequently, the fluid channel 515 can provide fluid communication between the interior of the fluid barrel 211 and the one or more fluid ports 106.

As can be appreciated from FIG. 5, the manifold sealing member 315 has formed a substantially fluid-tight seal with the manifold 105. Consequently, any fluid within the syringe assembly 107, and more specifically, the fluid barrel 211 and the fluid channel 515, is directed to the fluid port 106 and is substantially prevented from reaching other areas of the manifold 105.

Also visible in FIG. 5 is the fluid-tight sealing between the second end 318 of the plunger tip 316 and the fluid barrel 211. Consequently, fluid within the barrel 211 is substantially prevented from reaching the right side of the plunger tip 316 according to the orientation shown in the figures.

According to the embodiment shown in FIG. 5, the manifold 105 is fully engaged with the pump assembly housing 103 via the coupling tabs 208 and the grooves 209. The engagement between the manifold 105 and the pump assembly housing 103 brings the faces of the two housings into contact with one another. The coupling can also pull the syringe cartridge 107 against the plunger contact 445. More specifically, the coupling brings the plunger button 213 into contact with the plunger contact 445. Therefore, the plunger assembly 330 is forced to the fully extended (left) position because the actuation rod 440 is held in the fully extended position by the pump's actuation system. Therefore, as the manifold 105 is pulled to the right by the engagement between the grooves 209 and the coupling tabs 208, the syringe cartridge's components are pushed to the left due to interference of the pump assembly housing 103 and the syringe housing 210. This movement helps press the manifold sealing member 315 to form the fluid-tight seal with the manifold 105 as movement of the plunger assembly 330 is limited by the contact between the plunger tip 316 and the manifold sealing member 315.

Additionally, with the plunger assembly 330 being forced to the fully extended position as the syringe cartridge 107 is being installed into the syringe system 100, a user can be confident that accurate fluid dispensing is maintained from one syringe cartridge to another. This is because as the syringe cartridge 107 is installed, an initial “home” position is established. The precise positioning of the plunger contact 445, as discussed above, ensures that the plunger button 213 is flush with the manifold/pump housing interface when at the home position. Consequently, a user is not required to reprogram actuation of the plunger assembly 330 to account for changes in the plunger assembly's location. Rather, once manifold 105 is coupled to the pump housing 103, the pump assembly 101 can be actuated to move the syringe assembly 330 to the right to draw fluid into the fluid barrel and then actuated to the left to dispense fluid as needed.

In addition to the precise positioning of the plunger assembly 330, the present embodiment also prevents damage to the pump's electronics in the event of a plunger leak. According to an embodiment, the syringe system 100 provides an auxiliary flow path 500 that redirects the fluid thereby preventing the fluid from reaching the electronics in the event of a plunger failure. According to an embodiment, the auxiliary flow path 500 starts between the plunger rod 212 and the fluid barrel 211. As can be seen, there is an auxiliary flow channel 512 between the two components as the second end 318 of the plunger tip 316 comprises a larger diameter than the plunger rod 212. According to an embodiment, the plunger guide 320 includes one or more flow apertures 520. The flow apertures 520 are in fluid communication with the auxiliary flow channel 512 between the plunger rod 212, the fluid barrel 211, and the syringe housing 210. According to an embodiment, the manifold 105 further comprises one or more flow apertures 505, which can be in fluid communication with the flow apertures 520 formed in the plunger guide 320. The flow apertures 505 can further be in fluid communication with the environment, i.e., exposed to the outside. As can be seen, the sealing members 314 and 321 are positioned between the flow apertures 505 and 520 and the pump assembly 101. Consequently, in the event of a leak that escapes to the right side of the plunger tip 316, the fluid is diverted through the auxiliary flow path and substantially prevented from reaching the pump assembly 101 and any electronics of the pump assembly.

In use, the syringe cartridge 107 can be replaced to extend the life of the syringe system 100. The syringe cartridge 107 may be replaced due to a seal failure, after a predetermined time, after a predetermined number of actuation cycles, when the syringe is empty, etc.

According to an embodiment, the syringe cartridge 107 can be removed by disengaging the manifold 105 from the pump housing 103. In some embodiments, the disengagement may comprise rotating the manifold 105 with respect to the pump housing 103 to disengage the coupling tabs 208 from the grooves 209. In some embodiments, disengaging the manifold 105 from the pump housing 103 will also decouple the plunger button 213 from the plunger contact 445. For example, in some embodiments, the frictional fit between the syringe cartridge 107 and the manifold 105 will be stronger than the magnetic attraction between the plunger button 213 and the plunger contact 445. Therefore, the entire syringe cartridge 107 will be removed when the manifold 105 is disengaged. In other embodiments, disengaging the manifold 105 will leave the syringe 107 behind while being coupled to the plunger contact 445. If the syringe cartridge 107 is left behind, i.e., disengages from the manifold 105, the user can simply apply a threshold force to the syringe 107 to decouple the plunger button 213 from the plunger contact 445.

With the syringe cartridge 107 removed from the syringe system 100, a replacement syringe cartridge 107 can be provided. Prior to installing the replacement syringe cartridge 107, a user should ensure that the plunger contact 445 and thus, the actuation rod 440 are in the fully extended home position, i.e., the plunger contact 445 is flush with the face of the housing 103. According to an embodiment, once the plunger contact 445 is flush with the face of the housing 103, the replacement syringe cartridge 107 can be installed.

According to an embodiment, the replacement syringe cartridge 107 can be positioned proximate the plunger contact 445. For example, a user can position the plunger button 213 proximate the plunger contact 445. In some embodiments, the magnetic attraction between the two components may pull the plunger button 213 into contact with the plunger contact 445. With the syringe cartridge 107 in position, the manifold 105 can be slid over the syringe cartridge 107 and can be coupled to the pump assembly housing 103 via the coupling tabs 208 and the grooves 209, for example. The positioning of the plunger contact 445 and the actuation rod 440 ensure that when the manifold 105 is being coupled to the pump assembly housing 103, that the syringe cartridge 107 is pressed into the manifold 105 to form the substantially fluid-tight seal between the manifold 105 and the manifold sealing member 315.

According to another embodiment, rather than positioning the syringe cartridge 107 proximate the plunger contact 445 prior to engaging the manifold 105, the syringe cartridge 107 can first be at least partially inserted into the manifold 105. With the syringe cartridge 107 inserted into the manifold 105, the manifold 105 can then be coupled to the pump assembly housing 103. As discussed above, the engagement between the manifold 105 and the pump assembly housing 103 pushes the syringe cartridge 107 fully into the manifold 105 to ensure the fluid-tight seal.

The embodiments described above provide a reusable syringe system 100 that can accurately and quickly exchange syringe cartridges. The syringe system 100 incorporates a new cartridge assembly 102 that can be coupled to a pump assembly 101 to accurately position a syringe cartridge 107. By accurately positioning the components, precise fluid control can be achieved. As discussed above, many prior art systems are not able to accurately position the actuation rod and thus, the plunger assembly 330. However, the embodiments described above utilize a plunger contact 445 that is coupled to the actuation rod 440 to ensure a flush alignment at an end position. The flush alignment translates into a flush positioning of the plunger assembly 330 when the manifold 105 is coupled to the pump housing 103.

Additionally, the embodiments described above provide an auxiliary flow path that diverts fluid away from the pump assembly 101 in the event of a leak developing past the plunger tip 316. The auxiliary flow path can also provide a vent during normal use to prevent a vacuum from building up within the fluid barrel 211 as the plunger assembly 330 is being actuated.

The detailed descriptions of the above embodiments are not exhaustive descriptions of all embodiments contemplated by the inventors to be within the scope of the present description. Indeed, persons skilled in the art will recognize that certain elements of the above-described embodiments may variously be combined or eliminated to create further embodiments, and such further embodiments fall within the scope and teachings of the present description. It will also be apparent to those of ordinary skill in the art that the above-described embodiments may be combined in whole or in part to create additional embodiments within the scope and teachings of the present description.

Thus, although specific embodiments are described herein for illustrative purposes, various equivalent modifications are possible within the scope of the present description, as those skilled in the relevant art will recognize. The teachings provided herein can be applied to other syringe systems, and not just to the embodiments described above and shown in the accompanying figures. Accordingly, the scope of the embodiments described above should be determined from the following claims.

We claim:

1. A syringe system (100), comprising:
a pump assembly (101) including a movable actuation rod (440) and a plunger contact (445) coupled to an end of the movable actuation rod (440);
a cartridge assembly (102) including:
a manifold (105) removably coupled to the pump assembly (101); and
a syringe cartridge (107) positioned within the manifold (105) and including a plunger assembly (330) movable with respect to the manifold (105) upon actuation of the actuation rod (440).

2. The syringe system (100) of claim 1, wherein the syringe cartridge (107) further comprises a syringe housing (210) and a fluid barrel (211) positioned at least partially within the syringe housing (210).

3. The syringe system (100) of claim 2, wherein the plunger assembly (330) comprises a plunger button (213) contacting the plunger contact (445), a plunger rod (212) coupled to the plunger button (213), and a plunger tip (316) coupled to the plunger rod (212), with the plunger tip (316) forming a substantially fluid-tight seal with the fluid barrel (211).

4. The syringe system (100) of claim 3, further comprising a manifold sealing member (315) coupled to the fluid barrel (211) and forming a substantially fluid-tight seal with the manifold (105) and the syringe housing (210).

5. The syringe system (100) of claim 4, further comprising a fluid channel (515) formed in the manifold sealing member (315) and in fluid communication with one or more fluid ports (106) formed in the manifold (105).

6. The syringe system (100) of claim 3, further comprising a plunger guide (320) received by the syringe housing (210) and slidably receiving the plunger rod (212) to maintain an axial alignment of the plunger assembly (330).

7. The syringe system (100) of claim 6, further comprising an auxiliary flow path (500) defined by an auxiliary flow channel (512) between the plunger rod (212) and the fluid barrel (211), a fluid port (520) formed in the plunger guide (320), and a fluid port (505) formed in the manifold (105).

8. The syringe system (100) of claim 1, further comprising one or more coupling tabs (208) formed on the pump assembly (101) and one or more corresponding grooves (209) formed in the manifold (105).

9. The syringe system (100) of claim 1, further comprising a ramped surface (444) coupled to the actuation rod (440) positioned to contact a pump housing (103) at an extended stroke position.

10. The syringe system (100) of claim 9, wherein the plunger contact (445) is positioned such that the plunger contact (445) is substantially flush with the pump housing (103) when the actuation rod (440) is at the extended stroke position.

11. The syringe system (100) of claim 1, further comprising a position sensor (442) configured to determine at least an extended position of the actuation rod (440).

12. The syringe system (100) of claim 1, wherein the position sensor (442) comprises an optical position sensor and includes a sensor flag (441) coupled to the actuation rod (440).

13. The syringe system (100) of claim 1, wherein the plunger contact (445) comprises a magnet.

14. A cartridge assembly (102) for a syringe system (100), comprising:
a manifold (105) including one or more fluid ports (106);
a syringe housing (210) positioned within the manifold (105);
a fluid barrel (211) positioned within the syringe housing (210) and in fluid communication with the one or more fluid ports (106); and
a plunger assembly (330) movable within the fluid barrel (211) to draw fluid into and expel fluid out of the fluid barrel (211).

15. The cartridge assembly (102) of claim 14, wherein the plunger assembly (330) comprises a plunger tip (316), a plunger rod (212), and a plunger button (213), wherein the plunger button (213) is substantially flush with the manifold (105) when the plunger assembly (330) is in a fully extended position.

16. The cartridge assembly (102) of claim 15, further comprising a plunger guide (320) received by the syringe housing (210) and receives the plunger rod by a sliding radial contact to maintain an axial alignment of the plunger assembly (330).

17. The cartridge assembly (102) of claim 16, further comprising an auxiliary fluid path (500) defined by an auxiliary flow channel (512) between the plunger assembly (330) and the fluid barrel (211), a fluid port (520) formed in the plunger guide (320), and a fluid port (505) formed in the manifold (105).

18. The cartridge assembly (102) of claim 14, further comprising a manifold sealing member (315) coupled to the fluid barrel (211) and forming a substantially fluid-tight seal with the manifold (105) and the syringe housing (210).

19. The cartridge assembly (102) of claim 18, further comprising a fluid channel (515) formed in the manifold sealing member (315) and in fluid communication with one or more fluid ports (106) formed in the manifold (105).

20. A method for forming a syringe system, comprising steps of:
positioning a movable actuation rod and a plunger contact within a pump assembly;
positioning a syringe cartridge within a manifold, the syringe cartridge comprising a plunger assembly movable with respect to the manifold; and
removably coupling the manifold to the pump assembly such that actuation of the actuation rod moves the plunger assembly.

21. The method of claim 20, wherein the step of positioning the syringe cartridge comprises inserting a fluid barrel into a syringe housing and coupling the fluid barrel to the manifold to create a seal.

22. The method of claim 21, further comprising a step of aligning a fluid channel formed in the fluid barrel with one or more fluid ports formed in the manifold.

23. The method of claim 21, wherein the plunger assembly comprises a plunger tip forming a substantially fluid-tight seal with the fluid barrel, a plunger rod coupled to the plunger tip, and a plunger button coupled to the plunger rod, wherein the step of removably coupling the manifold positions the plunger button in contact with the plunger contact.

24. The method of claim 23, further comprising a step of positioning a plunger guide within the syringe housing and by a sliding radial contact, positions the plunger rod within the plunger guide to maintain axial alignment of the plunger assembly.

25. The method of claim 24, further comprising a step of defining an auxiliary flow channel between the plunger rod and the fluid barrel, which is in fluid communication with a fluid port formed in the plunger guide and a fluid port formed in the manifold.

26. The method of claim 20, wherein the step of removably coupling the manifold comprises engaging one or more coupling tabs extending from the pump assembly with one or more corresponding grooves formed in the manifold.

27. The method of claim 20, wherein the step of positioning the movable actuation rod comprises actuating the actuation rod to an extended position and coupling the plunger contact to the actuation rod such that the plunger contact is substantially flush with a housing of the pump assembly.

28. The method of claim 20, further comprising a step of coupling a portion of a position sensor to the actuation rod to detect at least an extended position of the actuation rod.
